(19) United States
(12) Patent Application Publication
(10) Pub. No.: US 2012/0208208 A1
(43) Pub. Date: Aug. 16, 2012

(54) PCSK9 IMMUNOASSAY

(76) Inventors: Yan G. Ni, Westfield, NJ (US); Shilpa Pandit, Edison, NJ (US)

(21) Appl. No.: 13/503,708

(22) PCT Filed: Oct. 28, 2010
(86) PCT No.: PCT/US10/54376

§ 371 Date: Apr. 24, 2012

Related U.S. Application Data
(60) Provisional application No. 61/256,688, filed on Oct. 30, 2009.

(51) Int. Cl. G01N 33/573

(52) U.S. Cl. 435/7.4

(57) ABSTRACT

Methods of using PCSK9 antagonists. More specifically, methods for measuring circulating PCSK9 levels in a biological sample by means of an immunoassay.

### BACKGROUND OF THE INVENTION

Proprotein convertase subtilisin-kexin type 9 (PCSK9), also known as neural apoptosis-regulated convertase 1 (NARC-1), is a proteinase K-like subtilase identified as the 9^{th} member of the secretory subtilase family (Seidah, N. G., et al., 2003 PROC NATL ACAD SCI USA 100:928-933). PCSK9 is expressed in cells capable of proliferation and differentiation such as hepatocytes, kidney mesenchymal cells, intestinal ileum, colon epithelia and embryonic brain telencephalic neurons (Seidah et al., 2003).

The gene for human PCSK9 has been sequenced and found to be about 22-kb long with 12 exons that encode a 692 amino acid protein (NP_{—}777596.2). PCSK9 is disclosed and/or claimed in several patent publications, including: PCT Publication Nos. WO 01/31007, WO 01/57081, WO 02/14358, WO 01/98468, WO 02/102993, WO 02/102994, WO 02/46383, WO 02/90526, WO 01/77137, and WO 01/34768; US Publication Nos. US 2004/0009553 and US 2003/0119038, and European Publication Nos. EP 1 440 981, EP 1 067 182, and EP 1 471 152.

PCSK9 has been implicated in cholesterol homeostasis, as it appears to have a specific role in cholesterol biosynthesis or uptake. In a study of cholesterol-fed rats, Maxwell et al. found that PCSK9 was downregulated in a similar manner to other genes involved in cholesterol biosynthesis, (Maxwell et al., 2003 J. LIPID RES. 44:2109-2119). The expression of PCSK9 was regulated by sterol regulatory element-binding proteins (SREBP), which is seen in other genes involved in cholesterol metabolism (Maxwell, et al., 2003).

Additionally, PCSK9 expression is upregulated by statins in a manner attributed to the cholesterol-lowering effects of the drugs (Dubuc et al., 2004 ARTERIOSCLER. THROMB. VASC. BIOL. 24:1454-1459). Adenoviral expression of PCSK9 has been shown to lead to a notable time-dependent increase in circulating low density lipoprotein (LDL) (Benjannet et al., 2004 J. BIOL. CHEM. 279:48865-48875) and mice with PCSK9 gene deletions have increased levels of hepatic LDL receptors (LDLR) and clear LDL from the plasma more rapidly (Rashid et al., 2005 PROC. NATL. ACAD. SCI. USA 102:5374-5379). Medium from HepG2 cells transiently transfected with PCSK9 reduce the amount of cell surface LDLRs and internalization of LDL when transferred to untransfected HepG2 cells (Cameron et al., 2006 HUMAN MOL. GENET. 15:1551-1558). It has been further demonstrated that purified PCSK9 added to the medium of HepG2 cells had the effect of reducing the number of cell-surface LDLRs in a dose- and time-dependent manner (Lagace et al., 2006 J. CLIN. INVEST. 116:2995-3005).

A number of mutations in the gene PCSK9 have also been conclusively associated with autosomal dominant hypercholesterolemia (ADH), an inherited metabolism disorder characterized by marked elevations of low density lipoprotein (“LDL”) particles in the plasma which can lead to premature cardiovascular failure (e.g., Abifadel et al., 2003 NATURE GENETICS 34:154-156; Timms et al., 2004 HUM. GENET. 114:349-353; Leren, 2004 CLIN. GENET. 65:419-422).

It therefore appears that PCSK9 plays a role in the regulation of LDL production. Expression or upregulation of PCSK9 is associated with increased plasma levels of LDL cholesterol, and inhibition or the lack of expression of PCSK9 is associated with low LDL cholesterol plasma levels. Significantly, lower levels of LDL cholesterol associated with sequence variations in PCSK9 confer protection against coronary heart disease (Cohen, et al., 2006 N. ENGL. J. MED. 354:1264-1272).

Clinical trial data has demonstrated that reductions in LDL cholesterol levels are related to the rate of coronary events (Law et aL, 2003 BMJ 326:1423-1427). Moderate lifelong reduction in plasma LDL cholesterol levels has been shown to be substantially correlated with a substantial reduction in the incidence of coronary events (Cohen et al., 2006, supra), even in populations with a high prevalence of non-lipid-related cardiovascular risk factors. Accordingly, there is great benefit to be reaped from the managed control of LDL cholesterol levels.

Accordingly, it would be desirable to further investigate PCSK9 as a target for the treatment of cardiovascular disease, Antibodies useful as PCSK9 antagonists have been identified and have utility as therapeutic agents. In support of such investigations, it would be useful to have a method for measuring levels of circulating PCSK9 in a biological sample which has been exposed to a PCSK9 antagonist, such as an antibody.

It would be further desirable to be able to identify novel PCSK9 antagonists in order to assist in the quest for compounds and/or agents effective in the treatment of cardiovascular disease. Hence, a method for measuring levels of circulating PCSK9 in a biological sample for such purposes as, e.g., assessing the effectiveness of a putative PCSK9 antagonist is desirable.

Additionally, it would be of use to provide kits to assay levels of circulating PCSK9 in biological samples.

### SUMMARY OF THE INVENTION

The present invention relates to a method of measuring circulating PCSK9 levels in a biological sample. Said method comprises the steps of performing an immunoassay on a biological sample obtained from a subject and comparing the level of PCSK9 in said sample against a standard having a known concentration of PCSK9.

The present invention further relates to a method for identifying novel PCSK9 antagonists, comprising the steps of performing an immunoassay on a biological sample which has been contacted with a putative PCSK9 antagonist and comparing the level of PCSK9 in said sample against a standard having a known concentration of PCSK9.

A further aspect of the present invention relates to a kit for measuring circulating PCSK9 levels in a biological sample, wherein said kit comprises:

a). a biological sample collection device;

b). a composition comprising an immunoassay which comprises a coating or capture antibody and a detection antibody;

and c). a means for detecting a reaction between PCSK9 antigen in the sample and antibodies in the immunoassay.

### BRIEF DESCRIPTION OF THE DRAWINGS

FIG. 1 illustrates a murine PCSK9 DELFIA assay.

FIG. 2 illustrates a dilution curve demonstrating plasma tolerance of murine serum/plasma obtained using the DELFIA murine plasma assay.

FIG. 3 illustrates circulating PCSK9 levels in C57B6 mice using the murine 1H23-1A08 PCSK9 DELFIA assay.

### DETAILED DESCRIPTION OF THE INVENTION

The present invention relates to a method of measuring circulating PCSK9 levels in a biological sample, comprising the steps of performing an immunoassay on a biological sample obtained from a subject and comparing the level of PCSK9 in said sample against a standard having a known concentration of PCSK9. The present assay is of particular utility for measuring murine PCSK9, an important criteria in evaluating animal and more particularly murine models.

An immunoassay is an analysis or methodology that utilizes an antibody to specifically bind an analyte. The immunoassay is characterized by the use of specific binding properties of at least one particular antibody to isolate, target or quantify the analyte.

In particular embodiments, the immunoassay comprises the steps of: (a) depositing a biological sample on a support having immobilized bound anti-PCSK9 antibody 1H23 bound thereto; (b) contacting the support having the biological sample deposited thereon with anti-PCSK9 antibody 1A08 bearing a detectable label; and (c) detecting the label.

PCSK9 refers to proprotein convertase subtilisin-kexin type 9 (PCSK9), also known as neural apoptosis-regulated convertase 1 (NARC-1), a proteinase K-like subtilase identified as the 9^{th} member of the secretory subtilase family (Seidah, N. G., et al., 2003 PROC NATL ACAD SCI USA 100:928-933), as defined in the literature and, unless otherwise stated, includes both the soluble and insoluble forms. The term may in appropriate context refer to either an antigenic component thereof or the genetic locus.

1H23 is an antibody molecule comprising a variable light (“VL”) sequence comprising SEQ ID NO: 13 and a variable heavy (“VH”) sequence comprising SEQ ID NO: 14. In particular embodiments, 1H23 is a full length antibody molecule. In specific embodiments, 1H23 is an IgG antibody molecule. In specific embodiments, 1H23 comprises (a) light chain comprising SEQ ID NO: 3 and (b) a heavy chain comprising SEQ ID NO: 4.

1A08 is an antibody molecule comprising a variable light (“VL”) sequence comprising SEQ ID NO: 15 and a variable heavy (“VH”) sequence comprising SEQ ID NO: 16. In particular embodiments, 1A08 is an antibody fragment. In specific embodiments, 1H23 is a Fab. In specific embodiments, 1H23 comprises (a) light chain comprising SEQ ID NO: 7 and (b) a heavy chain comprising SEQ ID NO: 8 exclusive of the c-myc and His tags noted in Example 1, and optionally containing one or more of said tags.

Antibody molecules can exist, for example, as intact immunoglobulins or as a number of well characterized fragments produced by, for example, digestion with various peptidases. The recognized immunoglobulin genes include the kappa, lambda, alpha, gamma, delta, epsilon and mu constant region genes, as well as a myriad of immunoglobulin variable region genes. Light chains are classified as gamma, mu, alpha, delta, or epsilon, which in turn define the immunoglobulin classes, IgG, IgM, IgA, IgD and IgE, respectively. “Whole” antibodies or “full length” antibodies often refers to proteins that comprise two heavy (H) and two light (L) chains inter-connected by disulfide bonds which comprise: (1) in terms of the heavy chains, a variable region (abbreviated herein as “V_{H}”) and a heavy chain constant region which comprises three domains, C_{H1}, C_{H2}, and C_{H3}; and (2) in terms of the light chains, a light chain variable region (abbreviated herein as “V_{L}”) and a light chain constant region which comprises one domain, C_{L}. Pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region thereby converting the F(ab)′₂ dimer into an Fab′ monomer. The Fab′ monomer is essentially a Fab with part of the hinge region broken. While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

In specific embodiments, the 1H23 and 1A08 antibody molecules are, independently, isolated prior to use. “Isolated”, as used herein, refers to a property that makes them different from that found in nature. The difference can be, for example, that they are of a different purity than that found in nature, or that they are of a different structure or form part of a different structure than that found in nature. A structure not found in nature, for example, includes recombinant human immunoglobulin structures. Other examples of structures not found in nature are antibody molecules substantially free of other cellular material.

A detectable label, as used herein, refers to another molecule or agent incorporated into or affixed to the antibody molecule. In one embodiment, the label is a detectable marker, e.g., a radiolabeled amino acid or attachment to a polypeptide of biotinyl moieties that can be detected by marked avidin (e.g., streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionuclides( e.g., ³H, ¹⁴C, ¹⁵N, 35S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin, and analogs or homologs thereof. In some embodiments, labels are attached by spacer arms of various lengths to reduce potential steric hindrance.

In particular embodiments of the present invention, the immunoassay is a solid phase immunoassay. In specific embodiments, the solid phase immunoassay is a dissociation-enhanced lanthanide fluorescence immunoassay (DELFIA). However, it is within the scope of the current invention to use any solution-based or solid phase immunoassay as will be well familiar to those of skill in the art. Such assays include, without limitation, assays using magnetic beads as labels in lieu of enzymes, ELISAs, radioisotopes, or fluorescent moieties (fluorescent immunoassays).

The biological sample is selected from the group consisting of blood, plasma and serum. Preferred subjects are mice.

The present invention further relates to a method for measuring PCSK9 in the presence of a putative PCSK9 antagonist. Said method comprises the steps of performing an immunoassay on a biological sample which has been contacted with a putative PCSK9 antagonist and comparing the level of PCSK9 in said sample against a standard having a known concentration of PCSK9. In particular embodiments, the method comprises (a) depositing the biological sample on a support having immobilized anti-PCSK9 antibody 1H23; (b) contacting the support having the biological sample deposited thereon with anti-PCSK9 antibody 1 A08 bearing a detectable label; (c) detecting the label; and (d) comparing the level of PCSK9 in said sample against a standard having a known concentration of PCSK9. In a preferred embodiment, the immunoassay is a solid phase immunoassay. In a more preferred embodiment, the solid phase immunoassay is a dissociation-enhanced lanthanide fluorescence immunoassay (DELFIA).

The biological sample is selected from the group consisting of blood, plasma and serum. Preferred subjects are mice.

Use of the term “antagonist” or derivatives thereof (e.g., “antagonizing”) refers to the fact that the subject molecule or agent can antagonize, oppose, counteract, inhibit, neutralize, or curtail the functioning of PCSK9. In specific embodiments, the antagonist reduces the functioning or activity or PCSK9 by at least 10%, or at least 20%, 30%, 40%, 50%, 60%, 70%, 80%, 90%, or 95%. Reference herein to PCSK9 function or PCSK9 activity refers to any function or activity that is driven by, requires, or is exacerbated or enhanced by PCSK9.

The present invention additionally relates to a kit for measuring circulating PCSK9 levels in a biological sample, comprising:

a). a biological sample collection device;

b). a composition comprising an immunoassay which comprises a coating or capture antibody and a detection antibody;

and c). a means for detecting a reaction between PCSK9 antigen in the sample and antibodies in the immunoassay; wherein the coating or capture antibody is 1H23 and the detecting antibody is 1A08.

In particular embodiments, the kit comprises the 1 H23 antibody immobilized on a support.

Kits typically but need not include a label indicating the intended use of the contents of the kit. The term label in the context of the kit includes any writing, or recorded material supplied on or with the kit, or which otherwise accompanies the kit.

The following examples are provided to illustrate the present invention without limiting the same hereto:

### Example 1

### PCSK9 Antagonists 1H23 & 1A08

The PCSK9 antagonists used in this assay are antibodies 1H23 and 1A08. 1H23 is disclosed in copending application Ser. No. 61/121,951, filed Dec. 12, 2008, which is incorporated in its entirety herein.

### Isolation of Recombinant Fab Display Phage 1H23 & 1A08

Recombinant Morphosys HuCAL Gold Fab phage display libraries (see, e.g., Knappik et al., 2000 J. Mol. Biol. 296:57-86; Rothe et al., 2008 J. Mol. Biol. 376:1182-1200) were panned against immobilized recombinant murine PCSK9 (1A08) and alternate pairings of human and murine PCSK9 (human/murine/human; 1H23) through a process which is briefly described as follows:

For the panning giving rise to 1H23, human and mouse PCSK9 protein were chemically biotinylated (Pierce, Cat. #21455) per manufacturer's instruction. The Morphosys phage Fab display libraries were pooled and pre-absorbed three times to blocked strepavidin coated beads (Dynal beads M280). The first and third panning rounds utilized human PCSK9, and the second panning round was directed against mouse PCSK9.

For each of the three rounds of panning, the preabsorbed phage library was incubated with preblocked biotinylated PCSK9 (150 nM for first round and 100 nM for subsequent rounds) immobilized to strepavidin coated Dynal beads. The immobilized phage-PCSK9 complexes were washed sequentially with 5 quick washes with PBS/0.05% Tween™ 20 followed by 4 quick washes with PBS and transferred in PBS to a fresh blocked tube. Bound phages were then eluted with 20 mM DTT. TG1 cells were infected with eluted phages. Pooled cultures of phagemid-bearing cells (chloramphenicol-resistant) were grown up and frozen stocks of phagernid-bearing cultures were made. Phage were rescued from culture by co-infection with helper phage, and phage stocks for next round of panning were made.

After the third round of panning phagemid-infected cells were grown overnight and phagemid DNA was prepared.

For the isolation of 1A08, three rounds of panning were performed against non-biotinylated murine PCSK9 immobilized on Maxisorp plates. Phage libraries were panned against immobilized recombinant human PCSK9 through a process which is briefly described as follows: Phage Fab display libraries were first divided into 3 pools: one pool of VH230 VH4+VH5, another of VH1+VH6, and a third pool of VH3. The phage pools and immobilized PCSK9 protein were blocked with nonfat dry milk.

For the first round of panning, each phage pool was bound independently to V5-, His-tagged murine PCSK9 protein immobilized in wells of Nunc Maxisorp plate. Immobilized phage-PCSK9 complexes were washed sequentially with (1) PBS/0.5% Tween™ 20 (Three quick washes); (2) PBS/0.5% Tween™ 20 (One 5 min. incubation with mild shaking); (3) PBS (Three quick washes); and (4) PBS (Two 5-min. incubations with mild shaking). Bound phages were eluted with 20 mM DTT and all three eluted phage suspensions were combined into one tube. E. coil TG1 were infected with eluted phages. Pooled culture of phagemid-bearing cells (chloramphenicol-resistant) were grown up and frozen stock of phagemid-bearing culture were made. Phage were rescued from culture by co-infection with helper phage, and phage stock for next round of panning were made.

For the second round of panning, phages from Round 1 were bound to immobilized, blocked V5-, His-tagged murine PCSK9 protein. Immobilized phage-PCSK9 complexes were washed sequentially with (1) PBS/0.05% Tween™ 20 (One quick wash); (2) PBS/0.05% Tween™ 20 (Four 5 min. incubations with mild shaking); (3) PBS (One quick wash); and (4) PBS (Four 5-min. incubations with mild shaking). Bound phages were eluted, E. coli TG1 cells were infected, and phage were rescued as in Round 1.

For the third round of panning, phages from Round 2 were bound to immobilized, blocked VS-His-tagged murine PCSK9 protein. Immobilized phage-PCSK9 complexes were washed sequentially with (1) PBS/0.05% Tween™ 20 (Ten quick washes); (2) PBS/0.05% Tween™ 20 (Five 5 min. incubations with mild shaking); (3) PBS (Ten quick washes); and (4) PBS (Five 5-min. incubations with mild shaking). Bound phages were eluted and E. coli TG1 cells were infected as in Round 1. Phagemid-infected cells were grown overnight and phagemid DNA was prepared.

XbaI-EcoRI inserts from Round 3 phagemid DNA were subcloned into Morphosys Fab expression vector pMORPH_x9_MH, and a library of Fab expression clones was generated in E. coli TG1 F-. Transformants were spread on LB+chloramphenicol+glucose plates and grown overnight to generate bacterial colonies. Individual transformant colonies were picked and placed into wells of two 96-well plates for growth and screening for Fab expression.

### ELISA Screening of Bacterially Expressed Fabs

Cultures of individual transformants were IPTG-induced and grown overnight for Fab expression. Culture supernatants (candidate Fabs) were incubated with purified V5-, His-tagged human or murine PCSK9 protein immobilized in wells of 96-well Nunc Maxisorp plates, washed with 0.1% Tween™ 20 in PBS using a plate washer, incubated with HRP-coupled anti-Fab antibody, and washed again with PBS/Tween™ 20. Bound HRP was detected by addition of TMP substrate, and A₄₅₀ values of wells were read with a plate reader.

Negative controls were included as follows:

Controls for nonspecific Fab binding on each plate were incubated with parallel expressed preparations of anti-EsB, an irrelevant Fab.
Growth medium only.

Positive controls for ELISA and Fab expression were included as follows: EsB antigen was bound to three wells of the plate and subsequently incubated with anti-EsB Fab. To control for Fabs reacting with the V5 or His tags of the recombinant PCSK9 antigen, parallel ELISAs were performed using V5-, His-tagged secreted alkaline phosphatase protein (SEAP) expressed in the same cells as the original PCSK9 antigen and similarly purified. Putative PCSK9-reactive Fabs were identified as yielding >3× background values when incubated with PCSK9 antigen but negative when incubated with SEAP. Clones scoring as PCSK9-reactive in the first round of screening were consolidated onto a single plate, re-grown in triplicate, re-induced with IPTG, and re-assayed in parallel ELISAs vs. PCSK9 and SEAP. Positive and negative controls were included as described above. Clones scoring positive in at least 2 of 3 replicates were carried forward into subsequent characterizations. In cases of known or suspected mixed preliminary clones, cultures were re-purified by streaking for single colonies on 2×YT plates with chloramphenicol, and liquid cultures from three or more separate colonies were assayed again by ELISAs in triplicate as described above.

### DNA Sequence Determination of PCSK9 ELISA-Positive Fab Clones

Bacterial cultures for DNA preps were made by inoculating 1.2 ml 2×YT liquid media with chloramphenicol from master glycerol stocks of positive Fabs, and growing overnight. DNA was prepared from cell pellets centrifuged out of the overnight cultures using the Qiagen Turbo Mini preps performed on a BioRobot 9600. ABI Dye Terminator cycle sequencing was performed on the DNA with Morphosys defined sequencing primers and run on an ABI 3100 Genetic Analyzer, to obtain the DNA sequence of the Fab clones. DNA sequences were compared to each other to determine unique clone sequences and to determine light and heavy chain subtypes of the Fab clones.

Expression and Purification of Fabs from Unique PCSK9 ELISA-Positive Clones

Fabs from ELISA-positive clones and the EsB (negative control) Fab were expressed by IPTG-induction in E. coli TGIF-cells. Cultures were lysed and the His-tagged Fabs were purified by immobilized metal ion affinity chromatography (IMAC), and proteins were exchanged into 25 mM HEPES pH 7.3/150 mM NaCl by centrifugal diafiltration. Proteins were analyzed by electrophoresis on Caliper Lab-Chip 90 and by conventional SDS-PAGE, and quantified by Bradford protein assay. Purified Fab protein was re-assayed by ELISA in serial dilutions to confirm activity of purified Fab. Positive and Negative controls were run as before. Purified Fab preparations were then analyzed as described below.

### Conversion of 1H23 Fab to Full Length IgG

The DNA sequence encoding the 1H23 light kappa chain variable region was amplified by polymerase chain reaction from plasmid template pMORPHx9_MH/PCSK9_{—}6_CX1_H23, using forward primer 5′-ACAGATGCCAGATGCGATATCGTGCTGACCCAGAG -3′ (SEQ ID NO: 9) and reverse primer 5′-CTTTGGCCTCTCTGGGATAGAAGTTATTCAGCAGGC-3′ (SEQ ID NO: 10). The product of this amplification was cloned into plasmid pV1JNSA-GS-FB-LCK that had been previously digested with FspI and BmtI, using the InFusion cloning system (Clontech). The resulting plasmid was verified by DNA sequencing across the variable region. Endotoxin-free plasmid preparations were made using the Qiagen Endo-Free plasmid maxiprep kit.

The DNA sequence encoding the heavy gamma chain variable region of pMORPHx9_MH/PCSK9_{—}6_CX1_H23 was amplified by polymerase chain reaction using forward primer 5′-ACAGGTGTCCACTCGCAGGTGCAATTGGTGGAAAGC-3′ (SEQ ID NO: 11) and reverse primer 5′-GCCCTTGGTGGATGCTGAGCTAACCGTCACCAGGGT-3′ (SEQ ID NO: 12), and the amplified product was cloned into plasmid pV1JNSA-BF-HCG2M4 that had been previously digested with FspI and BmtI. The resulting plasmid was verified by DNA sequencing across the variable region. Endotoxin-free plasmid preparations were made using the Qiagen Endo-Free plasmid maxiprep kit.

Full-length IgG was obtained by co-transfection of HEK293 cells with the 1H23 light chain- and heavy-chain-encoding plasmids, following by Protein A purification of the expressed IgG.

1H23 and 1A08 are characterized as follows:

```
1H23
SEQUENCES OF PCSK9_6_CX1_1123 IGG2M4 AS EXPRESSED
TRANSIENTLY IN HEK293 CELLS USING STANDARD
TRANSFECTION PROTOCOLS
6CX1H23 IgG Light Chain- VK3_3b (CDRs underlined in bold)
[SEQ ID NO: 1]
GATATCGTGCTGACCCAGAGCCCGGCGACCCTGAGCCTGTCTCCGGGCGAACGTGC
GACCCT
                CDR1
GAGCTGCAGAGCGAGCCAGTCTGTTAATTCTAATTATCTGGCTTGGTACCAGCAG
AAACC
                                  CDR2
AGGTCAAGCACCGCGTCTATTAATTTATGGTGCTTCTTCTCGTGCAACTGGGGTC
CCGGCGCGTTTTAGCGGCTCTGGATCCGGCACGGATTTTACCCTGACCATTAGCAGC
CTGGAACCTG
                                            CDR3
AAGACTTTGCGGTTTATTATTGCCAGCAGTGGGGTGATGTTCCTATTACCTTTGGC
CAGGGTACGAAAGTTGAAATTAAACGTACGGTGGCTGCACCATCTGTCTTCATCTTC
CCGCCATCTGATGAGCAGTTGAAATCTGGAACTGCCTCTGTTGTGTGCCTGCTGAAT
AACTTCTATCCCAGAGAGGCCAAAGTACAGTGGAAGGTGGATAACGCCCTCCAATC
GGGTAACTCCCAGGAGAGTGTCACAGAGCAGGACAGCAAGGACAGCACCTACAGCC
TCAGCAGCACCCTGACGCTGAGCAAAGCAGACTACGAGAAACACAAAGTCTACGCC
TGCGAAGTCACCCATCAGGGCCTGAGCTCGCCCGTCACAAAGAGCTTCAACAGGGG
AGAGTGT
VL of 6CX1H23
[SEQ ID NO: 17]
GATATCGTGCTGACCCAGAGCCCGGCGACCCTGAGCCTGTCTCCGGGCGAACGTGCGACCCT
GAGCTGCAGAGCGAGCCAGTCTGTTAATTCTAATTATCTGGCTTGGTACCAGCAGAAACCAG
GTCAAGCACCGCGTCTATTAATTTATGGTGCTTCTTCTCGTGCAACTGGGGTCCCGGCGCGTT
TTAGCGGCTCTGGATCCGGCACGGATTTTACCCTGACCATTAGCAGCCTGGAACCTGAAGAC
TTTGCGGTTTATTATTGCCAGCAGTGGGGTGATGTTCCTATTACCTTTGGCCAGGGTACGAA
AGTTGAAATTAAACGTACG
6CX1H23 IgG2m4 Heavy Chain- VH3_3 (CDRs underlined in bold)
[SEQ ID NO: 2]
CAGGTGCAATTGGTGGAAAGCGGCGGCGGCCTGGTGCAACCGGGCGGCAGCCTGCG
                                            CDR1
TCTGAGCTGCGCGGCCTCCGGATTTACCTTTTCTGATTATTATATGCATTGGGTGC
                                                            CDR2
GCCAAGCCCCTGGGAAGGGTCTCGAGTGGGTGAGCAATATCTCTGGTTCTGGTAG
CACTACCTATTATGCGGATAGCGTGAAAGGCCGTTTTACCATTTCACGTGATAATT
CGAAAAACACCCTGTATCTGCAAATGAACAGCCTGCGTGCGGAAGATACGGCCGTG
                                  CDR3
TATTATTGCGCGCGTGGTATGTTTGATTTTTGGGGCCAAGGCACCCTGGTGACGGT
TAGCTCAGCATCCACCAAGGGCCCATCCGTCTTCCCCCTGGCGCCCTGCTCCAGGAG
CACCTCCGAGAGCACAGCCGCCCTGGGCTGCCTGGTCAAGGACTACTTCCCCGAACC
GGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGACCTCCA
GCAACTTTGGCACGCAGACCTACACCTGCAACGTAGATCACAAGCCCAGCAACACC
AAGGTGGACAAGACAGTTGAGCGGAAATGCTGCGTGGAGTGCCCACCATGCCCAGC
ACCTCCAGTGGCCGGACCATCAGTCTTCCTGTTCCCCCCAAAACCCAAGGACACTCT
CATGATCTCCCGGACCCCTGAGGTCACGTGCGTGGTGGTGGACGTGAGCCAGGAAG
ACCCCGAGGTCCAGTTCAACTGGTACGTGGATGGCGTGGAGGTGCATAATGCCAAG
ACAAAGCCGCGGGAGGAGCAGTTCAACAGCACGTTCCGTGTGGTCAGCGTCCTCAC
CGTCCTGCACCAGGACTGGCTGAACGGCAAGGAGTACAAGTGCAAGGTCTCCAACA
AAGGCCTCCCGTCCTCCATCGAGAAAACCATCTCCAAAACCAAAGGGCAGCCCCGA
GAGCCACAGGTGTACACCCTGCCCCCATCCCGGGAGGAGATGACCAAGAACCAGGT
CAGCCTGACCTGCCTGGTCAAAGGCTTCTACCCCAGCGACATCGCCGTGGAGTGGG
AGAGCAATGGGCAGCCGGAGAACAACTACAAGACCACGCCTCCCATGCTGGACTCC
GACGGCTCCTTCTTCCTCTACAGCAAGCTAACCGTGGACAAGAGCAGGTGGCAGCA
GGGGAATGTCTTCTCATGCTCCGTGATGCATGAGGCTCTGCACAACCACTACACACA
GAAGAGCCTCTCCCTGTCTCCTGGTAAA
VH of 6CX1H23
[SEQ ID NO: 18]
CAGGTGCAATTGGTGGAAAGCGGCGGCGGCCTGGTGCAACCGGGCGGCAGCCTGCGTCTGA
GCTGCGCGGCCTCCGGATTTACCTTTTCTGATTATTATATGCATTGGGTGCGCCAAGCCCCTG
GGAAGGGTCTCGAGTGGGTGAGCAATATCTCTGGTTCTGGTAGCACTACCTATTATGCGGAT
AGCGTGAAAGGCCGTTTTACCATTTCACGTGATAATTCGAAAAACACCCTGTATCTGCAAAT
GAACAGCCTGCGTGCGGAAGATACGGCCGTGTATTATTGCGCGCGTGGTATGTTTGATTTTT
GGGGCCAAGGCACCCTGGTGACGGTTAGCTCA
6CX1H23 IgG Light Chain- VK3_3h (CDRs underlined in bold)
[SEQ ID NO: 3]
                                            CDR1 CDR2
DIVLTQSPATLSLSPGERATLSCRASQSVNSNYLAWYQQKPGQAPRLLIYGASSRATGV
                                                                CDR3
PARFSGSGSGTDFILTISSLEPEDFAVYYCQQWGDVPITFGQGTKVEIKRTVAAPSVFIFP
PSDEQLKSGTASVVCLLNNFYPREAKVQWKVDNALQSGNSQESVTEQDSKDSTYSLSST
LTLSKADYEKHKVYACEVTHQGLSSPVTKSFNRGEC
VL of 6CX1H23
[SEQ ID NO: 13]
DIVLTQSPATLSLSPGERATLSCRASQSVNSNYLAWYQQKPGQAPRLLIYGASSRATGVPARFSG
SGSGTDFTLTISSLEPEDFAVYYCQQWGDVPITFGQGTKVEIKRT
6CX1H23 IgG2m4 Heavy Chain- VH3_3 (CDRs underlined in bold)
[SEQ ID NO: 4]
                                                CDR1 CDR2
QVQLVESGGGLVQPGGSLRLSCAASGFTFSDYYMHWVRQAPGKGLEWVSNISGSGST
                                                                                  CDR3
TYYADSVKGRFTISRDNSKNTLYLQMNSLRAEDTAVYYCARGMFDFWGQGTLVTVSS
ASTKGPSVFPLAPCSRSTSESTAALGCLVKDYFPEPVTVSWNSGALTSGVHTFPAVLQSS
GLYSLSSVVTVTSSNFGTQTYTCNVDHKPSNTKVDKTVERKCCVECPPCPAPPVAGPSV
FLFPPKPKDTLMISRTPEVTCVVVDVSQEDPEVQFNWYVDGVEVHNAKTKPREEQFNST
FRVVSVLTVLHQDWLNGKEYKCKVSNKGLPSSIEKTISKTKGQPREPQVYTLPPSREEMT
KNQVSLTCLVKGFYPSDIAVEWESNGQPENNYKTTPPMLDSDGSFFLYSKLTVDKSRWQ
QGNVFSCSVMHEALHNHYTQKSLSLSPGK
VH of 6CX1H23
[SEQ ID NO: 14]
QVQLVESGGGLVQPGGSLRLSCAASGFTFSDYYMHWVRQAPGKGLEWVSNISGSGSTTYYADS
VKGRFTISRDNSKNTLYLQMNSLRAEDTAVYYCARGMFDFWGQGTLTVSS
1A08
SEQUENCES OF PCSK9_2_CX1_A08 Fab AS EXPRESSED FROM MORPHOSYS ™
RECOMBINANT Fab DISPLAY PHAGE LIBRARY IN E coli
2CX1A08 Fab Light Chain- VK1_3 (CDRs underlined in bold)
[SEQ ID NO: 5]
GATATCCAGATGACCCAGAGCCCGTCTAGCCTGAGCGCGAGCGTGGGTGATCGTGT
                          CDR1
GACCATTACCTGCAGAGCGAGCCAGGATATTTCTAATTATCTGACTTGGTACCAG
                                                  CDR2
CAGAAACCAGGTAAAGCACCGAAACTATTAATTTATGCTGCTTCTTCTTTGCAAA
GCGGGGTCCCGTCCCGTTTTAGCGGCTCTGGATCCGGCACTGATTTTACCCTGACCA
                                                                                  CDR3
TTAGCAGCCTGCAACCTGAAGACTTTGCGACTTATTATTGCTTTCAGTTTGATAATG
TTCCTCTTACCTTTGGCCAGGGTACGAAAGTTGAAATTAAACGTACGGTGGCTGCTC
CGAGCGTGTTTATTTTTCCGCCGAGCGATGAACAACTGAAAAGCGGCACGGCGAGC
GTGGTGTGCCTGCTGAACAACTTTTATCCGCGTGAAGCGAAAGTTCAGTGGAAAGTA
GACAACGCGCTGCAAAGCGGCAACAGCCAGGAAAGCGTGACCGAACAGGATAGCA
AAGATAGCACCTATTCTCTGAGCAGCACCCTGACCCTGAGCAAAGCGGATTATGAA
AAACATAAAGTGTATGCGTGCGAAGTGACCCATCAAGGTCTGAGCAGCCCGGTGAC
TAAATCTTTTAATCGTGGCGAGGCC
VL of 2CX1A08
[SEQ ID NO: 19]
GATATCCAGATGACCCAGAGCCCGTCTAGCCTGAGCGCGAGCGTGGGTGATCGTGTGACCA
TTACCTGCAGAGCGAGCCAGGATATTTCTAATTATCTGACTTGGTACCAGCAGAAACCAGGT
AAAGCACCGAAACTATTAATTTATGCTGCTTCTTCTTTGCAAAGCGGGGTCCCGTCCCGTTTT
AGCGGCTCTGGATCCGGCACTGATTTTACCCTGACCATTAGCAGCCTGCAACCTGAAGACTT
TGCGACTTATTATTGCTTTCAGTTTGATAATGTTCCTCTTACCTTTGGCCAGGGTACGAAAGT
TGAAATTAAACGTACG
2CX1A08 Fab Heavy Chain- VH5_3 (CDRs underlined in bold;
c-myc tag underlined in bold and italics; His tag
underlined, not bold)
[SEQ ID NO: 6]
CAGGTGCAATTGGTTCAGAGCGGCGCGGAAGTGAAAAAACCGGGCGAAAGCCTGA
A
                                            CDR1
AATTAGCTGCAAAGGTTCCGGATATTCCTTTTCTACTTATTGGATTGGTTGGGTGC
                                                    CDR2
GCCAGATGCCTGGGAAGGGTCTCGAGTGGATGGGCATTATCGATCCGGGTGATA
GCTTTACCCGTTATTCTCCGAGCTTTCAGGGCCAGGTGACCATTAGCGCGGATAA
AAGCATTAGCACCGCGTATCTTCAATGGAGCAGCCTGAAAGCGAGCGATACGGCCA
                                    CDR3
TGTATTATTGCGCGCGTGGTTATCATGATGAGCCTTATGGTTTTTTTGATGTTTGG
GGCCAAGGCACCCTGGTGACGGTTAGCTCAGCGTCGACCAAAGGTCCAAGCGTGTT
TCCGCTGGCTCCGAGCAGCAAAAGCACCAGCGGCGGCACGGCTGCCCTGGGCTGCC
TGGTTAAAGATTATTTCCCGGAACCAGTCACCGTGAGCTGGAACAGCGGGGCGCTG
ACCAGCGGCGTGCATACCTTTCCGGCGGTGCTGCAAAGCAGCGGCCTGTATAGCCTG
AGCAGCGTTGTGACCGTGCCGAGCAGCAGCTTAGGCACTCAGACCTATATTTGCAAC
GTGAACCATAAACCGAGCAACACCAAAGTGGATAAAAAAGTGGAACCGAAAAGCG
AATTC [symbol] [symbol] [symbol] [symbol] [symbol] GGCGCGCCGCACCATCATC
ACCATCAC
VH of 2CX1A08
[SEQ ID NO: 20]
CAGGTGCAATTGGTTCAGAGCGGCGCGGAAGTGAAAAAACCGGGCGAAAGCCTGAAAATTA
GCTGCAAAGGTTCCGGATATTCCTTTTCTACTTATTGGATTGGTTGGGTGCGCCAGATGCCTG
GGAAGGGTCTCGAGTGGATGGGCATTATCGATCCGGGTGATAGCTTTACCCGTTATTCTCCG
AGCTTTCAGGGCCAGGTGACCATTAGCGCGGATAAAAGCATTAGCACCGCGTATCTTCAATG
GAGCAGCCTGAAAGCGAGCGATACGGCCATGTATTATTGCGCGCGTGGTTATCATGATGAG
CCTTATGGTTTTTTTGATGTTTGGGGCCAAGGCACCCTGGTGACGGTTAGCTCA
2CX1A08 Fab Light Chain- VK1_3 (CDRs underlined in bold)
[SEQ ID NO: 7]
                                            CDR1 CDR2
DIQMTQSPSSLSASVGDRVTITCRASQDISNYLTWYQQKPGKAPKLLIYAASSLQSGVP
S
                                                        CDR3
RFSGSGSGTDFTLTISSLQPEDFATYYCFQFDNVPLTFGQGTKVEIKRTVAAPSVFIFPPSD
EQLKSGTASVVCLLNNEYPREAKVQWKVDNALQSGNSQESVTEQDSKDSTYSLSSTLTL
SKADYEKHKVYACEVTHQGLSSPVTKSFNRGEA
VL of 2CX1A08
[SEQ ID NO: 15]
DIQMTQSPSSLSASVGDRVTITCRASQDISNYLTWYQQKPGKAPKLLIYAASSLQSGVPSRFSGSG
SGTDFTLTISSLQPEDFATYYCFQFDNVPLTFGQGTKVEIKRT
2CX1A08 Fab Heavy Chain- VH5_3 (CDRs underlined in bold;
c-myc tag underlined in bold and italics; His tag underlined,
not bold)
[SEQ ID NO: 8]
                                              CDR1 CDR 2
QVQLVQSGAEVKKPGESLKISCKGSGYSFSTYWIGWVRQMPGKGLEWMGIIDPGDSF
                                                                                  CDR3
TRYSPSFQGQVTISADKSISTAYLQWSSLKASDTAMYYCARGYHDEPYGFEDVWGQG
TLVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSEF [symbol] [symbol]
NGAPHHHHHH
VH of 2CX1A08
[SEQ ID NO: 16]
QVQLVQSGAEVKKPGESLKISCKGSGYSFSTYWIGWVRQMPGKGLEWMGIIDPGDSFTRYSPSF
QGQVTISADKSISTAYLQWSSLKASDTAMYYCARGYHDEPYGFFDVWGQGTLVTVSS
```

### Example 2

### Solid Phase Immunoassay (DELFIA)

96-well plates (high-binding 4HBX plates from Thermo Labsystems, part # 3855) were coated overnight at 4° with 50 μl of 10 μg/ml of anti-PCSK9 antibody (6CX1H23IgG), the coating/capture antibody. 6CX1H23 binds both human and mouse PCSK9, as well as rat and hamster. H23 has also been used as a detection antibody for rhesus target engagement (measurement of Total PCSK9). The next day, the wells were blocked with 250 μl of blocking solution (1% BSA (KPL) in TBS (BIORAD) with 0.05% Tween-20) for 1 hour at room temperature. Plates were washed in a plate-washer with wash buffer (imidazole buffered saline with Tween 20 (KPL)). For the standard, purified mouse PCSK9 protein was titrated starting at 1 μg/ml, with a 2-fold titration in diluent (1% BSA in PBS). Purified mouse PCSK9 protein was diluted in assay buffer (1% BSA in PBS) and 100 μl of dilute protein was added on the plate as standard. Plates were incubated at 37° for 2 hours. Plates were again washed in a plate-washer with wash buffer.

Subsequently, the detection step was carried out. 100 μl of 1 μg/ml of biotinylated anti-PCSK9 Fab (1A08) was added on the plates as the primary or capture antibody. 2CX1A08 is specific for mouse PCSK9. After the plates were washed, 75 μl of 1:1000 Streptavidin/Europium (Perkin Elmer, part # 1244-360) (diluted in assay buffer) was added. The plates were then incubated at room temperature for 20 minutes. The plates were washed again followed by the addition of 100 μl of DELFIA Enhance solution (Perkin Elmer part # 1244-105) in order to enhance the fluorescence. The europium fluorescence was measured using a Europium plate reader after one hour.

The sensitivity of this assay is ˜100 pM with a signal to noise ratio of about 2-fold.

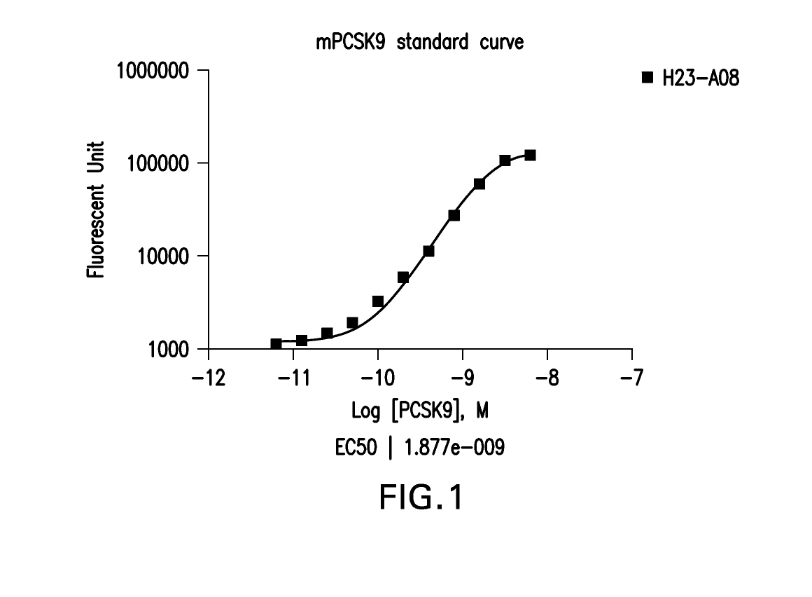

As shown in FIG. 1, PCSK9 levels range from 10 pm to 10 nM in these samples.

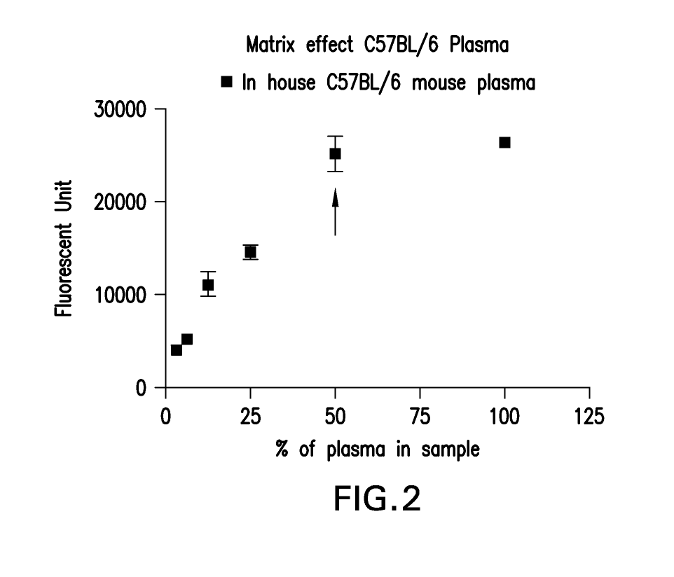

FIG. 2 illustrates a dilution curve demonstrating plasma tolerance obtained with the DELFIA murine plasma assay. Here, PCSK9 levels from healthy mice were tested in the murine PCSK9 DELFIA assay using the 1H23-1A08 format. Due to the limitation of sample volume, murine plasma samples were diluted 8 fold before testing. Results are mean±SD, n=3. Murine plasma sample was diluted with assay buffer (1% BSA in PBS) and then assayed in PCSK9 DELFIA using the 1H23-1A08 format. As shown in FIG. 2, this assay can tolerate up to 50% of murine serum or plasma sample.

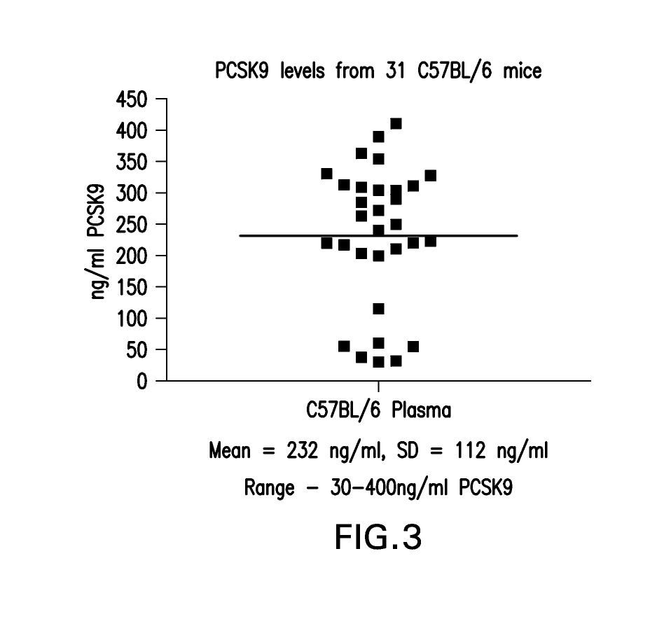

As shown in FIG. 3, PCSK9 levels in murine plasma samples were diluted 8 fold and assessed in thirty-one C57/B6 mice with the 1H23-1A08 format. The PCSK9 levels range from 30-400 ng/ml, with a mean value of 232 ng/ml.

What is claimed is:

1. A method of measuring circulating PCSK9 levels in a biological sample comprising the steps of performing an immunoassay on a biological sample obtained from a subject and comparing the level of PCSK9 in said sample against a standard having a known concentration of PCSK9, wherein a coating or capture antibody is 1H23 and a detecting antibody is 1A08.

2. The method of claim 1 wherein 1H23 is a full length antibody and 1 A08 is a Fab.

3. The method of claim 1 wherein 1H23 comprises a variable light (“VL”) sequence comprising SEQ ID NO: 13 and a variable heavy (“VH”) sequence comprising SEQ ID NO: 14, and 1A08 comprises a variable light (“VL”) sequence comprising SEQ ID NO: 15 and a variable heavy (“VH”) sequence comprising SEQ ID NO: 16.

4. The method of claim 1 wherein 1H23 comprises a light chain comprising SEQ ID NO: 3 and a heavy chain comprising SEQ ID NO: 4 and 1A08 comprises (a) light chain comprising SEQ ID NO: 7 and (b) a heavy chain comprising SEQ ID NO: 8 exclusive of the c-myc and His tags, and optionally containing one or more of said tags.

5. The method of claim 1 wherein performing an immunoassay comprises: (a) depositing a biological sample on a support having immobilized anti-PCSK9 antibody 1H23; (b) contacting the support having the biological sample deposited thereon with anti-PCSK9 antibody 1A08 bearing a detectable label; and (c) detecting the label.

6. The method of claim 1, wherein the immunoassay is a solid phase immunoassay.

7. The method of claim 6, wherein the solid phase immunoassay is a dissociation-enhanced lanthanide fluorescence immunoassay (DELFIA).

8. The method of claim 1, wherein said sample is selected from the group consisting of blood, plasma and serum.

9. The method of claim 8 wherein the blood, plasma or serum is from a mouse.

10. A method for performing an immunoassay on a biological sample which has been contacted with a putative PCSK9 antagonist which comprises (a) depositing the biological sample on a support having immobilized anti-PCSK9 antibody 1H23; (b) contacting the support having the biological sample deposited thereon with anti-PCSK9 antibody 1A08 bearing a detectable label; (c) detecting the label; and (d) comparing the level of PCSK9 in said sample against a standard having a known concentration of PCSK9.

11. The method of claim 10 wherein 1H23 is a full length antibody and 1A08 is a Fab.

12. The method of claim 10 wherein 1H23 comprises a variable light (“VL”) sequence comprising SEQ ID NO: 13 and a variable heavy (“VH”) sequence comprising SEQ ID NO: 14, and 1A08 comprises a variable light (“VL”) sequence comprising SEQ ID NO: 15 and a variable heavy (“VH”) sequence comprising SEQ ID NO: 16.

13. The method of claim 10 wherein 1 H23 comprises a light chain comprising SEQ ID NO: 3 and a heavy chain comprising SEQ ID NO: 4 and 1A08 comprises (a) light chain comprising SEQ ID NO: 7 and (b) a heavy chain comprising SEQ ID NO: 8 exclusive of the c-myc and His tags, and optionally containing one or more of said tags.

14. The method of claim 10, wherein the immunoassay is a solid phase immunoassay.

15. The method of claim 14, wherein the solid phase immunoassay is a dissociation-enhanced lanthanide fluorescence immunoassay (DELFIA).

16. The method of claim 10, wherein said sample is selected from the group consisting of blood, plasma and serum.

17. The method of claim 16 wherein the blood, plasma or serum is from a mouse.

18. A kit for measuring circulating PCSK9 levels in a biological sample, comprising:
a). a biological sample collection device;
b). a composition comprising an immunoassay which comprises a coating or capture antibody and a detection antibody; and
c). a means for detecting a reaction between PCSK9 antigen in the sample and antibodies in the immunoassay;
wherein the coating or capture antibody is 1H23 and the detecting antibody is 1A08.

19. The kit of claim 18 wherein 1H23 is a full length antibody and 1A08 is a Fab.

20. The method of claim 19 wherein 1H23 comprises a variable light (“VL”) sequence comprising SEQ ID NO: 13 and a variable heavy (“VH”) sequence comprising SEQ ID NO: 14, and 1A08 comprises a variable light (“VL”) sequence comprising SEQ ID NO: 15 and a variable heavy (“VH”) sequence comprising SEQ ID NO: 16.

21. The method of claim 20 wherein 1H23 comprises a light chain comprising SEQ ID NO: 3 and a heavy chain comprising SEQ ID NO: 4 and 1A08 comprises (a) light chain comprising SEQ ID NO: 7 and (b) a heavy chain comprising SEQ ID NO: 8 exclusive of the c-myc and His tags, and optionally containing one or more of said tags.


SEQUENCE LISTING (20 sequences)

SEQ ID NO: 1 - DNA, 645 bp, Artificial Sequence
  6CX1H23 IgG Antibody Light Chain
gatatcgtgctgacccagagcccggcgaccctgagcctgtctccgggcgaacgtgcgaccctgagctgcagagcgagccagtctgttaattctaattatctggcttggtaccagcagaaaccaggtcaagcaccgcgtctattaatttatggtgcttcttctcgtgcaactggggtcccggcgcgttttagcggctctggatccggcacggattttaccctgaccattagcagcctggaacctgaagactttgcggtttattattgccagcagtggggtgatgttcctattacctttggccagggtacgaaagttgaaattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 2 - DNA, 1320 bp, Artificial Sequence
  6CX1H23 IgG2m4 Antibody Heavy Chain
caggtgcaattggtggaaagcggcggcggcctggtgcaaccgggcggcagcctgcgtctgagctgcgcggcctccggatttaccttttctgattattatatgcattgggtgcgccaagcccctgggaagggtctcgagtgggtgagcaatatctctggttctggtagcactacctattatgcggatagcgtgaaaggccgttttaccatttcacgtgataattcgaaaaacaccctgtatctgcaaatgaacagcctgcgtgcggaagatacggccgtgtattattgcgcgcgtggtatgtttgatttttggggccaaggcaccctggtgacggttagctcagcatccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgacctccagcaactttggcacgcagacctacacctgcaacgtagatcacaagcccagcaacaccaaggtggacaagacagttgagcggaaatgctgcgtggagtgcccaccatgcccagcacctccagtggccggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgttccgtgtggtcagcgtcctcaccgtcctgcaccaggactggctgaacggcaaggagtacaagtgcaaggtctccaacaaaggcctcccgtcctccatcgagaaaaccatctccaaaaccaaagggcagccccgagagccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctaaccgtggacaagagcaggtggcagcaggggaatgtcttctcatgctccgtgatgcatgaggctctgcacaaccactacacacagaagagcctctccctgtctcctggtaaa

SEQ ID NO: 3 - protein, 215 aa, Artificial Sequence
  6CX1H23 IgG Antibody Light Chain
DIVLTQSPATLSLSPGERATLSCRASQSVNSNYLAWYQQKPGQAPRLLIYGASSRATGVPARFSGSGSGTDFTLTISSLEPEDFAVYYCQQWGDVPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 440 aa, Artificial Sequence
  6CX1H23 IgG2m4 Antibody Heavy Chain
QVQLVESGGGLVQPGGSLRLSCAASGFTFSDYYMHWVRQAPGKGLEWVSNISGSGSTTYYADSVKGRFTISRDNSKNTLYLQMNSLRAEDTAVYYCARGMFDFWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 5 - DNA, 642 bp, Artificial Sequence
  2CX1A08 Fab Antibody Light Chain
gatatccagatgacccagagcccgtctagcctgagcgcgagcgtgggtgatcgtgtgaccattacctgcagagcgagccaggatatttctaattatctgacttggtaccagcagaaaccaggtaaagcaccgaaactattaatttatgctgcttcttctttgcaaagcggggtcccgtcccgttttagcggctctggatccggcactgattttaccctgaccattagcagcctgcaacctgaagactttgcgacttattattgctttcagtttgataatgttcctcttacctttggccagggtacgaaagttgaaattaaacgtacggtggctgctccgagcgtgtttatttttccgccgagcgatgaacaactgaaaagcggcacggcgagcgtggtgtgcctgctgaacaacttttatccgcgtgaagcgaaagttcagtggaaagtagacaacgcgctgcaaagcggcaacagccaggaaagcgtgaccgaacaggatagcaaagatagcacctattctctgagcagcaccctgaccctgagcaaagcggattatgaaaaacataaagtgtatgcgtgcgaagtgacccatcaaggtctgagcagcccggtgactaaatcttttaatcgtggcgaggcc

SEQ ID NO: 6 - DNA, 735 bp, Artificial Sequence
  2CX1A08 Fab Antibody Heavy Chain
caggtgcaattggttcagagcggcgcggaagtgaaaaaaccgggcgaaagcctgaaaattagctgcaaaggttccggatattccttttctacttattggattggttgggtgcgccagatgcctgggaagggtctcgagtggatgggcattatcgatccgggtgatagctttacccgttattctccgagctttcagggccaggtgaccattagcgcggataaaagcattagcaccgcgtatcttcaatggagcagcctgaaagcgagcgatacggccatgtattattgcgcgcgtggttatcatgatgagccttatggtttttttgatgtttggggccaaggcaccctggtgacggttagctcagcgtcgaccaaaggtccaagcgtgtttccgctggctccgagcagcaaaagcaccagcggcggcacggctgccctgggctgcctggttaaagattatttcccggaaccagtcaccgtgagctggaacagcggggcgctgaccagcggcgtgcatacctttccggcggtgctgcaaagcagcggcctgtatagcctgagcagcgttgtgaccgtgccgagcagcagcttaggcactcagacctatatttgcaacgtgaaccataaaccgagcaacaccaaagtggataaaaaagtggaaccgaaaagcgaattcgagcagaagctgatctctgaggaggatctgaacggcgcgccgcaccatcatcaccatcac

SEQ ID NO: 7 - protein, 214 aa, Artificial Sequence
  2CX1A08 Fab Antibody Light Chain
DIQMTQSPSSLSASVGDRVTITCRASQDISNYLTWYQQKPGKAPKLLIYAASSLQSGVPSRFSGSGSGTDFTLTISSLQPEDFATYYCFQFDNVPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 8 - protein, 245 aa, Artificial Sequence
  2CX1A08 Fab Antibody Heavy Chain
QVQLVQSGAEVKKPGESLKISCKGSGYSFSTYWIGWVRQMPGKGLEWMGIIDPGDSFTRYSPSFQGQVTISADKSISTAYLQWSSLKASDTAMYYCARGYHDEPYGFFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFEQKLISEEDLNGAPHHHHHH

SEQ ID NO: 9 - DNA, 35 bp, Artificial Sequence
  Amplification primer
acagatgccagatgcgatatcgtgctgacccagag

SEQ ID NO: 10 - DNA, 36 bp, Artificial Sequence
  Amplification primer
ctttggcctctctgggatagaagttattcagcaggc

SEQ ID NO: 11 - DNA, 36 bp, Artificial Sequence
  Amplification primer
acaggtgtccactcgcaggtgcaattggtggaaagc

SEQ ID NO: 12 - DNA, 36 bp, Artificial Sequence
  Amplification primer
gcccttggtggatgctgagctaaccgtcaccagggt

SEQ ID NO: 13 - protein, 110 aa, Artificial Sequence
  VL of 6CX1H23 Antibody
DIVLTQSPATLSLSPGERATLSCRASQSVNSNYLAWYQQKPGQAPRLLIYGASSRATGVPARFSGSGSGTDFTLTISSLEPEDFAVYYCQQWGDVPITFGQGTKVEIKRT

SEQ ID NO: 14 - protein, 114 aa, Artificial Sequence
  VH of 6CX1H23 Antibody
QVQLVESGGGLVQPGGSLRLSCAASGFTFSDYYMHWVRQAPGKGLEWVSNISGSGSTTYYADSVKGRFTISRDNSKNTLYLQMNSLRAEDTAVYYCARGMFDFWGQGTLVTVSS

SEQ ID NO: 15 - protein, 109 aa, Artificial Sequence
  VL of 2CX1A08 Antibody
DIQMTQSPSSLSASVGDRVTITCRASQDISNYLTWYQQKPGKAPKLLIYAASSLQSGVPSRFSGSGSGTDFTLTISSLQPEDFATYYCFQFDNVPLTFGQGTKVEIKRT

SEQ ID NO: 16 - protein, 121 aa, Artificial Sequence
  VH of 2CX1A08 Antibody
QVQLVQSGAEVKKPGESLKISCKGSGYSFSTYWIGWVRQMPGKGLEWMGIIDPGDSFTRYSPSFQGQVTISADKSISTAYLQWSSLKASDTAMYYCARGYHDEPYGFFDVWGQGTLVTVSS

SEQ ID NO: 17 - DNA, 330 bp, Artificial Sequence
  VL of 6CX1H23 Antibody
gatatcgtgctgacccagagcccggcgaccctgagcctgtctccgggcgaacgtgcgaccctgagctgcagagcgagccagtctgttaattctaattatctggcttggtaccagcagaaaccaggtcaagcaccgcgtctattaatttatggtgcttcttctcgtgcaactggggtcccggcgcgttttagcggctctggatccggcacggattttaccctgaccattagcagcctggaacctgaagactttgcggtttattattgccagcagtggggtgatgttcctattacctttggccagggtacgaaagttgaaattaaacgtacg

SEQ ID NO: 18 - DNA, 342 bp, Artificial Sequence
  VH of 6CX1H23 Antibody
caggtgcaattggtggaaagcggcggcggcctggtgcaaccgggcggcagcctgcgtctgagctgcgcggcctccggatttaccttttctgattattatatgcattgggtgcgccaagcccctgggaagggtctcgagtgggtgagcaatatctctggttctggtagcactacctattatgcggatagcgtgaaaggccgttttaccatttcacgtgataattcgaaaaacaccctgtatctgcaaatgaacagcctgcgtgcggaagatacggccgtgtattattgcgcgcgtggtatgtttgatttttggggccaaggcaccctggtgacggttagctca

SEQ ID NO: 19 - DNA, 327 bp, Artificial Sequence
  VL of 2CX1A08 Antibody
gatatccagatgacccagagcccgtctagcctgagcgcgagcgtgggtgatcgtgtgaccattacctgcagagcgagccaggatatttctaattatctgacttggtaccagcagaaaccaggtaaagcaccgaaactattaatttatgctgcttcttctttgcaaagcggggtcccgtcccgttttagcggctctggatccggcactgattttaccctgaccattagcagcctgcaacctgaagactttgcgacttattattgctttcagtttgataatgttcctcttacctttggccagggtacgaaagttgaaattaaacgtacg

SEQ ID NO: 20 - DNA, 363 bp, Artificial Sequence
  VH of 2CX1A08 Antibody
caggtgcaattggttcagagcggcgcggaagtgaaaaaaccgggcgaaagcctgaaaattagctgcaaaggttccggatattccttttctacttattggattggttgggtgcgccagatgcctgggaagggtctcgagtggatgggcattatcgatccgggtgatagctttacccgttattctccgagctttcagggccaggtgaccattagcgcggataaaagcattagcaccgcgtatcttcaatggagcagcctgaaagcgagcgatacggccatgtattattgcgcgcgtggttatcatgatgagccttatggtttttttgatgtttggggccaaggcaccctggtgacggttagctca